(19) United States
(12) Patent Application Publication
(10) Pub. No.: US 2007/0025928 A1
(43) Pub. Date: Feb. 1, 2007

(54) Stannous oral care compositions

(75) Inventors: William Michael Glandorf, Mason, OH (US); Donald James White JR., Fairfield, OH (US); Arif A. Baig, Mason, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/541,889

(22) Filed: Oct. 2, 2006

Related U.S. Application Data
(63) Continuation-in-part of application No. 10/975,963, filed on Oct. 28, 2004.
Application No. 10/975,963 is a division of application No. 10/351,205, filed on Jan. 24, 2003, now Pat. No. 6,821,507.
Application No. 10/351,205 is a division of application No. 09/710,440, filed on Nov. 10, 2000, now Pat. No. 6,555,094.
(60) Provisional application No. 60/165,350, filed on Nov. 12, 1999.

(51) Int. Cl. A61K 8/21

(52) U.S. Cl. 424/49; 424/52

(57) ABSTRACT

Disclosed are oral compositions comprising a stannous ion source, a polyvalent cation source and a mineral surface active agent, said compositions providing enhanced therapeutic efficacy derived from stannous fluoride and/or other stannous salt, including antimicrobial effects, control of breath malodor, control of dental plaque growth and metabolism, reduced gingivitis, decreased progression to periodontal disease, reductions in dentinal hypersensitivity and reduced coronal and root dental caries. The aforementioned benefits are provided along with significant improvements compared to conventional stannous containing compositions, including: 1) reduced levels of dental staining; 2) reduced astringency thereby improving aesthetic characteristics of the compositions; 3) reduction in dental calculus formation, and 4) enhanced stability, bioavailability and thus, efficacy of stannous. The mineral surface active agents are agents that are substantive to mineral surfaces such as teeth and have chelating activity for polyvalent cations including stannous (Sn⁺²), zinc (Zn⁺²), copper (Cu⁺²), aluminum (Al⁺²), iron (Fe⁺², Fe⁺³), strontium (Sr⁺²), calcium (Ca⁺²), barium (Ba⁺²), magnesium (Mg⁺²), and manganese (Mn⁺²). Preferred mineral surface-active agents include polymers or copolymers containing phosphate, phosphonate, or carboxy groups. The compositions may also comprise a fluoride ion source and may be formulated as single phase or dual phase compositions.

### CROSS REFERENCE TO RELATED APPLICATIONS

This application is a continuation-in-part of U.S. application Ser. No. 10/975,963, filed Oct. 28, 2004, now pending, which is a divisional of U.S. application Ser. No. 10/351,205, filed Jan. 24, 2003 now U.S. Pat. No. 6,821,507, which is a divisional of U.S. application Ser. No. 09/710,440, filed Nov. 10, 2000 now U.S. Pat. No. 6,555,094 and which claims the benefit of U.S. Provisional Application No. 60/165350, filed Nov. 12, 1999.

### FIELD OF THE INVENTION

The present invention relates to improved oral compositions containing stannous salts, such as stannous fluoride. These improved compositions provide a spectrum of intraoral benefits derived from stannous fluoride and/or other stannous salt, including antimicrobial effects, control of breath malodor, control of dental plaque growth and metabolism, reduced gingivitis, decreased progression to periodontal disease, reductions in dentinal hypersensitivity, and reduced coronal and root dental caries and erosion. The aforementioned benefits are provided along with significant improvements compared to conventional stannous containing compositions, including: 1) reduced levels of dental staining; 2) reduced astringency thereby improving aesthetic characteristics of the compositions; 3) reduction in dental calculus formation, and 4) enhanced stability, bioavailability and thus, efficacy of stannous as antimicrobial, anti-plaque and anti-gingivitis agent. The improved stannous containing compositions provide these benefits through the combined effects of stannous, a second polyvalent cation and mineral surface-active and chelating agents, preferably polymeric and in particular including anionic polymers, such as condensed polyphosphate, polyphosphonate or polycarboxylate. The invention also relates to methods of enhancing therapeutic efficacy while decreasing staining and improving the aesthetic desirability of oral compositions containing stannous salts, such as stannous fluoride.

### BACKGROUND OF THE INVENTION

Stannous fluoride is commonly known for its efficacy when formulated into oral products. Stannous fluoride was the first fluoride source incorporated into toothpastes for therapeutic efficacy in the control of dental caries. Stannous fluoride gels, rinses, and dentifrices have since been shown to provide clinical efficacy for the reduction of dental caries, dentinal hypersensitivity, dental plaque and gingivitis. In addition to these clinical effects, formulations containing stannous fluoride may also help to provide improved breath benefits through chemical and antibacterial actions. Stannous fluoride formulations typically include stabilization systems designed to maintain bioavailable (i.e., soluble and reactive) levels of stannous during shelf storage, accounting for loss of stannous to oxidation or precipitation. Therefore, stannous fluoride formulations have been formulated with additional stannous containing ingredients, which provide a high concentration of stannous as a reservoir of stannous to maintain clinical efficacy. Unfortunately, although stannous fluoride compositions are known to be highly effective, successful commercial utilization is complicated by complexity in the development of formulations providing adequate stannous fluoride stability and in the side effects of stannous. Formulations providing increased or improved efficacy typically promote increased side effects. This limits clinical and commercial applications.

One of the most notable side effects of regular use of stannous fluoride is yellow-brown tooth staining. This stain is derived from pellicle, plaque and dietary component reactions with available stannous deposited on tooth surfaces during treatment with effective stannous fluoride formulations.

A second side effect routinely encountered during use of effective stannous fluoride formulations is unacceptable formulation astringency. Astringents are locally applied protein precipitants whose low cell permeability restricts actions to cell surfaces and interstitial spaces. Strong astringents can induce contraction and wrinkling of the tissues and mucous secretions can be precipitated or reduced. Within oral products, these chemical actions produce an unpleasant ‘drying’ sensation in the oral cavity, such as on the tongue, gingival tissues or buccal epithelia. Stannous formulations containing sufficient stannous for bioavailability are routinely described as astringent by patients and consumers and this property is undesirable. The astringency is most noticeable after use of the product.

A third side effect of the regular use of stannous fluoride dentifrice compositions is the decreased efficacy in reducing dental calculus with these compositions. It has been established that stannous fluoride dentifrices proven effective for antimicrobial, antigingivitis and other expected benefits do not always show reproducible clinical actions toward the prevention of accumulation of undesirable supragingival dental calculus. The control of supragingival calculus formation along with other clinical benefits is desired by professionals, patients and consumers. The multifunctional activity of oral compositions can simplify hygiene and provide a holistic approach to maintenance therapeutic oral health.

Previous attempts to develop effective and consumer acceptable stannous fluoride oral compositions have attempted to solve these cumulative detriments, however none have been fully successful. U.S. Pat. No. 5,004,597, issued to Majeti et al., discloses oral compositions containing stannous fluoride and gluconate salts. The inclusion of stannous gluconate results in improved formulation efficacy and stability. While effective, this formulation produces undesirable levels of tooth staining. Moreover, the formulation had unacceptable aesthetics, derived primarily from the astringency of stannous. Likewise, U.S. Pat. No. 5,578,293, issued to Prencipe et al., discloses the use of an organic acid compound to stabilize the stannous ion concentration. Coupled with the stannous fluoride and citrate as the organic acid, the formulations also include soluble pyrophosphate salts. U.S. Pat. No. 4,323,551 to Parran et al., discloses the use of pyrophosphate salts to provide anticalculus benefits. Clinical research has established the potential of anionic mineral surface-active inhibitors, such as pyrophosphates, in preventing the development of natural and antimicrobial induced tooth staining. (Grossman, Bollmer, Sturzenberger and Vick; Journal of Clinical Dentistry 6(4): 185-187, 1995). In the Prencipe et al. patent, all examples include sufficient amount of either citric acid and/or sodium citrate dihydrate to stabilize the stannous ions and to prevent precipitation. These levels also directly inhibit stannous binding to pyrophosphate salts. If stannous did bind to the pyrophosphate salts, studies support that this would decrease the antimicrobial activity of the stannous fluoride. The level of citrate needed to effectively stabilize the stannous ion against precipitation and pyrophosphate binding also significantly detracts from the aesthetics of the stannous composition. The composition will be salty, sour, and the stannous bound to citrate will still act as an astringent, which reduces the overall taste acceptability. U.S. Pat. No. 5,213,790, issued to Lukacovic et al., also discloses the use of a citrate ion source in a stannous composition. U.S. Pat. No. 5,780,015, issued to Fisher et al., discloses the use of dual phase dentifrice containing a potassium salt and a stannous salt wherein hydrogenated castor oil is used to help reduce astringency. The stannous salt is stabilized through the use of an organic acid compound as described in Prencipe et al. Another attempt to produce efficacious stannous composition is described in U.S. Pat. No. 5,716,600, issued to Zahradnik et al. This patent discloses low water formulations which help to prevent the stannous fluoride from degradation over time. No attempts are made to reduce the staining of the formulation.

U.S. Pat. No. 5,017,363, issued to Suhonen, discloses a stannous ion chelating copolymer of an alkyl vinyl ether and maleic anhydride or acid in an amount to effectively stabilize stannous ions. Suhonen also discloses that the compositions are substantially free from silica, soluble phosphates such as soluble pyrophosphates (e.g., tetrasodium pyrophosphate and tetrapotassium pyrophosphate), and aldehyde group containing compounds, since the stabilizing function of the stannous ion chelating polymer is not effective in the presence of these ingredients.

U.S. Pat. No. 5,338,537, issued to White, Jr. et al., discloses the use of a low molecular weight diphosphonic acid, which is used as a binding agent for stannous to help reduce the tendency of staining from the composition. While effective in reducing staining potential, laboratory studies have demonstrated that the antibacterial activity of formulations containing stannous complexed with the low molecular weight diphosphonic acid is very low. Similar results are obtained on formulation with soluble pyrophosphate salts, in the absence of strong citrate chelation, as described above.

Based on the foregoing, it appears that the same chemical and biochemical binding sites may be involved for both antibacterial/antiplaque activity and for stabilization and reducing the tooth staining potential of stannous fluoride. Thus, to achieve stabilization and/or reduction of tooth staining, antibacterial/antiplaque activity may be compromised. This makes the development of optimal stannous fluoride oral compositions difficult and explains the limited number of stannous fluoride compositions in the marketplace today. To improve consumer acceptance and compliance with the use of oral compositions containing stannous, a stannous composition is needed which has high efficacy but with low level of staining and other negative aesthetics, such as astringency. Moreover, it is desirable that these formulations provide simultaneous efficacy toward the reduction and control of dental calculus formation.

### SUMMARY OF THE INVENTION

The present invention relates to oral compositions comprising a stannous ion source, a polyvalent cation source and a mineral surface active agent that binds stannous, said compositions providing enhanced therapeutic efficacy with minimal side effects of tooth staining and astringency. The compositions simultaneously provide reduction and control of supragingival calculus. The mineral surface active agents are agents that are substantive to mineral surfaces such as teeth and additionally have chelating activity for polyvalent cations such as stannous (Sn⁺²), zinc (Zn⁺²), copper (Cu⁺²), aluminum (Al⁺³), iron (Fe⁺², Fe⁺³), strontium (Sr⁺²), calcium (Ca⁺²), barium (Ba⁺²), magnesium (Mg⁺²) and manganese (Mn⁺²). The compositions may also comprise a fluoride ion source. The present oral care compositions may be formulated as single phase or dual phase compositions. The present invention also provides a method for effective delivery of stannous-containing compositions with minimal side effects of tooth staining or astringency and with effective tartar control by administering to a subject a stable dentifrice composition comprising a clinically effective amount of stannous fluoride and/or other stannous salts in combination with a mineral surface active agent, preferably a phosphate- , phosphonate- or carboxy-containing polymer and a polyvalent cation source.

These and other features, aspects, and advantages of the present invention will become evident to those skilled in the art from the detailed description which follows.

### DETAILED DESCRIPTION OF THE INVENTION

While the specification concludes with claims, which particularly point out and distinctly claim the invention, it is believed the present invention will be better understood from the following description.

All percentages used herein are by weight of the dentifrice composition, unless otherwise specified. The ratios used herein are molar ratios of the overall composition, unless otherwise specified. All measurements are made at 25° C., unless otherwise specified.

Herein, “comprising” means that other steps and other ingredients which do not affect the end result can be added. This term encompasses the terms “consisting of” and “consisting essentially of”.

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The oral composition of the present invention may be in the form of a toothpaste, dentifrice, tooth powder, topical oral gel, mouthrinse, denture product, mouthspray, lozenge, oral tablet, or chewing gum.

The term “dentifrice”, as used herein, means paste, gel, or liquid formulations unless otherwise specified. The dentifrice composition may be in any desired form, such as deep striped, surface striped, multi-layered, having the gel surrounding the paste, or any combination thereof.

The oral composition may be a single phase oral composition or may be a combination of the two or more oral compositions, each in a separate phase. By “single or separate phase” herein is meant that all components of each composition are mixed together in one mixture which may contain liquid, solid and gaseous components. Thus each phase may be homogeneous or non-homogeneous. The oral composition is a product, which in the ordinary course of administration, is not intentionally swallowed for purposes of systemic administration of particular therapeutic agents, but is rather retained in the oral cavity for a time sufficient to contact substantially all of the tooth surfaces and/or oral tissues for purposes of oral activity. The term “total composition” as used herein means the total composition delivered to the oral cavity, regardless of whether it contains a single phase or multiple phases.

If a dual phase oral composition is desired, each oral composition will be contained in a physically separated compartment of a dispenser and dispensed side-by-side. The term “dispenser”, as used herein, means any pump, tube, or container suitable for dispensing toothpaste.

The term “orally acceptable carrier” as used herein includes safe and effective materials for use in the compositions of the present invention. Such materials are conventional additives in oral care compositions including but not limited to fluoride ion sources, anti-calculus or anti-tartar agents, buffers, abrasives such as silica, bleaching agents such as peroxide sources, alkali metal bicarbonate salts, thickening materials, humectants, water, surfactants, titanium dioxide, flavor system, sweetening agents, xylitol, coloring agents, and mixtures thereof.

Herein, the terms “tartar” and “calculus” are used interchangeably and refer to mineralized dental plaque biofilms.

The term “stannous” as used herein, is defined to mean the stannous that is in a dentifrice or other oral product, and supplied by a source such as stannous salts including stannous fluoride. It may refer to the stannous ions that are provided by a stannous salt other than stannous fluoride, added for stabilization purposes.

Active and other ingredients useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

The present invention relates to oral compositions comprising a stannous ion source, a source of polyvalent cations other than stannous and a mineral surface active agent having chelating activity for stannous and polyvalent cations, said compositions providing enhanced therapeutic efficacy from stannous with minimal side effects of tooth staining and astringency. By “therapeutic efficacy from stannous” herein is meant to include antimicrobial effects, control of breath malodor, control of dental plaque growth and metabolism, reduced gingivitis, decreased progression to periodontal disease, and reduction in dentinal hypersensitivity. The compositions preferably comprise a source of fluoride, which may be stannous fluoride, other fluoride salts or combinations thereof. The compositions simultaneously provide reduction and control of supragingival calculus. Reduction of tooth staining and astringency may also enhanced by concurrent appropriate formulation, including utilization of suitable poloxamer ingredients.

The mineral surface active agent (MSA) is preferably polymeric, in particular anionic polymers such as a polyphosphate having an average chain length of about 4 or more, a polyphosphonate, or other phosphate- or phosphonate- or carboxy-containing polymers. One having ordinary skill in the art would assume that a polymeric binding agent, such as a polyphosphate having an average chain length of about 4 or more, would behave similarly to the pyrophosphate or tripolyphosphate in stannous containing dentifrice systems. It has been found that chemical binding or chelation of stannous using pyrophosphate, diphosphonate, or tripolyphosphate to prevent stain formation, also produces unacceptable losses in therapeutic potential. However, an unexpected result occurs with longer-chain polyphosphates and other phosphate- or phosphonate-containing polymers as they are capable of reducing the side effects of dental staining and formulation astringency without significantly reducing the efficacy of the stannous. In fact including these polymeric MSA's in oral compositions containing stannous salts such as stannous fluoride, has been found to provide significant therapeutic efficacy with decreased levels of staining and astringency, while simultaneously providing reductions in supragingival calculus as compared to prior-art compositions containing stannous fluoride alone or stannous fluoride with stabilizing agents such as citrate.

The present oral care compositions may be formulated as single phase or dual phase compositions. One embodiment of the present invention provides a dual phase oral composition comprising a first composition comprising a stannous ion source and a source of polyvalent metal ions other than stannous, preferably zinc, and a second composition comprising a MSA. The MSA in this embodiment may be a linear polyphosphate having an average chain length of about 4 or more. The composition containing the polyphosphate will preferably have a limited water content up to about 20% to minimize hydrolysis of the polyphosphate.

A further embodiment of the present invention relates to a single phase oral composition comprising a stannous ion source, a source of polyvalent metal ions other than stannous and a MSA such as a linear polyphosphate having an average chain length of about 4 or more. The single-phase composition is formulated such that the linear polyphosphate is stabilized against hydrolytic degradation.

The present invention also relates to single phase or dual phase compositions comprising a stannous ion source, a source of polyvalent metal ions other than stannous and an anionic polymer of MW 500 or more containing one or combinations of phosphonate, diphosphonate, and carboxy functionalities.

The invention also provides a method for effective delivery of stannous-containing compositions with minimal side effects of tooth staining or astringency and with effective tartar control by administering to a subject a stable dentifrice composition comprising a clinically effective amount of stannous fluoride and/or other stannous salts in combination with a source of polyvalent metal ions other than stannous and a mineral surface-active and chelating agent, such as a phosphate- or phosphonate-containing polymer. One method for delivery of this improved stannous oral composition involves application of a dentifrice comprising two dentifrice compositions which are contained in physically separated compartments. Another method involves administering to a subject a stable single-phase dentifrice composition. One embodiment of a stable single phase composition comprises a polyphosphate, or other phosphate- or phosphonate-containing anionic polymer, stannous fluoride as a stannous ion source, a zinc ion source, wherein the composition may have a low total water content, depending upon stability requirements.

A preferred method for delivery of the present improved stannous-containing compositions involves application of a dentifrice comprising two dentifrice compositions which are contained in physically separated compartments. The physical separation allows for adequate stabilization of each dentifrice phase and ingredients therein. When combined in use, the chemical interactions of stannous (from stannous fluoride and/or other stannous salt) in one dentifrice phase with the MSA in a separate dentifrice phase allow appropriate delivery of both ingredients, thus, producing full therapeutic activity along with the provision of significant efficacy for the reduction of dental calculus and with marked reductions in undesirable side effects of tooth staining and astringency. The first dentifrice composition will comprise a source of stannous ions while the second dentifrice composition preferably comprises a polyphosphate or other anionic polymer or copolymer containing phosphate, phosphonate, carboxy groups or mixtures thereof. The source of polyvalent cations other than stannous may be incorporated in either or both first and second compositions, depending upon interactions with other components.

The essential and optional components of the compositions of the present invention are described in the following paragraphs.

Stannous Ion Sources

The present invention includes a stannous ion source as one essential component. The stannous ions are provided from stannous fluoride and/or other stannous salt that are added to the oral composition. Stannous fluoride has been found to help in the reduction of caries, gingivitis, plaque, and sensitivity, and in providing breath benefits. The stannous provided in the oral composition will provide efficacy to a subject using the composition. Other stannous salts include stannous chloride dihydrate, stannous acetate, stannous gluconate, stannous oxalate, stannous sulfate, stannous lactate, and stannous tartrate. The preferred stannous ion sources are stannous fluoride and stannous chloride dihydrate. The combined stannous salts will be present in an amount of from about 0.05% to about 11%, by weight of the total composition. Preferably, the stannous salts are present in an amount of from about 0.1 to about 7%, more preferably from about 0.4% to about 3%. Formulations typically include stannous levels, provided by stannous fluoride and other stannous salts, ranging from about 3,000 ppm to about 15,000 ppm stannous ions in the total composition.

Dentifrices containing stannous salts, particularly stannous fluoride and stannous chloride, are described in U.S. Pat. No. 5,004,597 to Majeti et al. Other descriptions of stannous salts are found in U.S. Pat. No. 5,578,293 issued to Prencipe et al. and in U.S. Pat. No. 5,281,410 issued to Lukacovic et al. In addition to the stannous ion source, other ingredients needed to stabilize the stannous may be included, such as the ingredients described in Majeti et al. and Prencipe et al.

Mineral Surface-Active Agent (MSA)

The present invention includes a mineral surface-active agent (MSA), which has substantivity to teeth and also has chelating or binding activity for stannous and other polyvalent cations. The abbreviation “MSA” herein designates such agents. The “mineral” descriptor is intended to convey that the surface activity or substantivity of the surface-active agent is toward mineral surfaces such as calcium phosphate minerals or teeth. Preferred MSA's are polymeric, in particular polymers containing anionic groups selected from phosphate, phosphonate, carboxy and mixtures thereof. Such anionic functionalities provide these agents with the capability to interact with cationic or positively charged entities.

These agents show affinity for binding stannous, in particular by stannous ion chelation, as evidenced by ionic fluoride release from stannous fluoride (SnF₂) and provision of increased ionic form of fluoride upon binding of the stannous. Effective agents also show surface reactivity toward calcium phosphate minerals, and are thus expected to retard calculus or tartar formation. The agents may also provide stain control, surface conditioning and antierosion benefits. Ideally, these agents will bind the stannous but will still enable the combined mixture to provide the desired tartar control, stain control, and surface conditioning, without having a negative effect on the efficacy of stannous fluoride for the control of dental caries, oral malodor and periodontal diseases including gingivitis.

Binding or chelating of stannous by the MSA provides a means of stabilizing stannous particularly in an aqueous environment, wherein stannous can form stannic compounds or can precipitate from solution, thereby reducing the amount of available stannous ions and consequently, the efficacy of the composition. For example, stannous fluoride compositions have been reported to be stabilized by a copolymer of an alkyl vinyl ether and maleic anhydride or acid in U.S. Pat. No. 5,017,363, to Suhonen. The copolymers are reported to have the ability to form chelates with the stannous ion which are sufficiently strong to prevent oxidation of stannous or precipitation from solution.

In addition to binding or chelating stannous, the preferred polymeric MSA's will have a strong affinity for enamel surface and will deposit a polymer layer or coating on the enamel surface and produce desired surface protection and conditioning effects. Suitable examples of such polymers are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); polycarboxylates and carboxy-substituted polymers; and mixtures thereof. Suitable polymeric mineral surface active agents include the carboxy-substituted alcohol polymers described in U.S. Pat. Nos. 5,292,501; 5,213,789, 5,093,170; 5,009,882; and 4,939,284; all to Degenhardt et al.; the diphosphonate-derivatized polymers in U.S. Pat. No. 5,011,913 to Benedict et al; and the synthetic anionic polymers including polyacrylates and copolymers of maleic anhydride or acid and methyl vinyl ether (e.g., Gantrez), as described, for example, in U.S. Pat. No. 4,627,977, to Gaffar et al. A preferred polymer is diphosphonate modified polyacrylic acid. Polymers with activity have sufficient surface binding propensity to desorb pellicle proteins and remain affixed to enamel surfaces. For tooth surfaces, polymers with end or side chain phosphate or phosphonate functions may both be preferred although other polymers with mineral binding activity may prove effective depending upon adsorption affinity.

Additional examples of suitable phosphonate containing polymeric MSA's include the geminal diphosphonate polymers disclosed as anticalculus agents in U.S. Pat. No. 4,877,603 to Degenhardt et al; phosphonate group containing copolymers disclosed in U.S. Pat. No. 4,749,758 to Dursch et al. and in GB 1,290,724 (both assigned to Hoechst) as suitable for use in detergent and cleaning compositions; and the copolymers and cotelomers disclosed as useful for applications including scale and corrosion inhibition, coatings, cements and ion-exchange resins in U.S. Pat. No. 5,980,776 to Zakikhani et al. and in U.S. Pat. No. 6,071,434 to Davis et al. Preferred polymers include the water-soluble copolymers of vinylphosphonic acid and acrylic acid and salts thereof disclosed in GB 1,290,724 wherein the copolymers contain from about 10% to about 90% by weight vinylphosphonic acid and from about 90% to about 10% by weight acrylic acid, more particularly wherein the copolymers have a weight ratio of vinylphosphonic acid to acrylic acid of 70% vinylphosphonic acid to 30% acrylic acid; 50% vinylphosphonic acid to 50% acrylic acid; or 30% vinylphosphonic acid to 70% acrylic acid. Other preferred polymers include the water soluble polymers disclosed by Zakikhani and Davis prepared by copolymerizing diphosphonate or polyphosphonate monomers having one or more unsaturated C═C bonds (e.g., vinylidene-1,1-diphosphonic acid and 2-(hydroxyphosphinyl)ethylidene-1,1-diphosphonic acid), with at least one further compound having unsaturated C═C bonds (e.g., acrylate and methacrylate monomers), such as those having the following structure:
1. Co-telomer of Acrylic Acid and 2-(hydroxyphosphinyl)ethylidene-1,1-diphosphonic Acid With Structure: 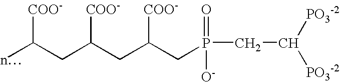
2. Co-Polymer of Acrylic Acid and vinyldiphosphonic Acid With Structure:

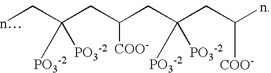

Suitable polymers include the diphosphonate/acrylate polymers supplied by Rhodia under the designation ITC 1087 (Average MW 3000-60,000) and Polymer 1154 (Average MW 6000-55,000).

A preferred MSA will be stable with other components of the formulation such as ionic fluoride and will not hydrolyze in high water content formulations, thus permitting a simple single phase dentifrice or mouthrinse formulation. If the MSA's does not have these stability properties, one option is to formulate a dual phase formulation with the MSA's physically separated from the incompatible component(s).

A preferred polymeric MSA is a polyphosphate. A polyphosphate is generally understood to consist of two or more phosphate molecules arranged primarily in a linear configuration, although some cyclic derivatives may be present. Particularly effective are polyphosphates having an average chain length of about four or more phosphate groups so that surface adsorption at effective concentrations produces sufficient non-bound phosphate functions which enhance the anionic surface charge as well as hydrophilic character of the surfaces. The pyrophosphates and tripolyphosphates are discussed separately under additional anticalculus agents. The longer-chain polyphosphate salts include tetrapolyphosphate and hexametaphosphate, among others. Polyphosphates larger than tetrapolyphosphate usually occur as amorphous glassy materials. Examples of suitable polyphosphates are the linear “glassy” polyphosphates having the formula:
XO(XPO₃)ₙX
wherein X is sodium, potassium or ammonium and n averages from about 6 to about 125. Preferred are polyphosphates manufactured by FMC Corporation which are commercially known as Sodaphos (n≈6), Hexaphos (n≈13), and Glass H (n≈21). The most preferred polyphosphate is Glass H. These polyphosphates may be used alone or in a combination thereof.

It is known that polyphosphates with an average chain length greater than about 4 will react with ionic fluoride in oral compositions at ambient temperature and produce monofluorophosphate ions, in addition to altering the pH of the composition. This reaction compromises the efficacy of the oral composition and its ability to provide stable ionic fluoride and polyphosphate to the oral surfaces. It is also known that polyphosphates undergo hydrolysis. Therefore, formulating with such polyphosphates presents some difficulties. One way to stabilize the polyphosphate from hydrolysis and/or interaction with incompatible ingredients is to reduce the total water content of the dentifrice composition. U.S. Pat. No. 5,939,052 issued to White, Jr. et al. further describes the polyphosphates. The phosphate sources are also described in more detail in Kirk-Othmer Encyclopedia of Chemical Technology, Fourth Edition, Volume 18, Wiley-Interscience Publishers (1996).

For compositions containing condensed polyphosphate having an average of 21 phosphate repeating units, an ideal the ratio of total moles of phosphate anion to total moles of stannous ion has been found to be a molar ratio of phosphate anion to stannous ion of from about 0.2:1 to about 5:1, preferably from about 0.5:1 to about 3:1, more preferably from about 0.6:1 to about 2:1, and most preferably from about 0.7:1 to about 1:1.

Other polyphosphorylated compounds may be used as the MSA/chelant in addition to or instead of the polyphosphate, in particular polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof. Preferred herein is phytic acid, also known as myo-inositol 1,2,3,4,5,6-hexakis (dihydrogen phosphate) or inositol hexaphosphoric acid, and its alkali metal, alkaline earth metal or ammonium salts. Herein, the term “phytate” includes phytic acid and its salts as well as the other polyphosphorylated inositol compounds.

Still other non-polymeric MSA's that may be used are chelating materials that have been suggested in the art for the purpose of retarding calculus formation and removing calculus after it is formed. The chemical approach to calculus inhibition generally involves chelation of calcium ion and/or crystal growth inhibition which prevents the calculus from forming and/or breaks down mature calculus by removing calcium. These chemical chelants are discussed in more detail below under the section on Anticalculus Agents.

The amount of MSA required is an effective amount which will bind the stannous, permit adequate antimicrobial activity, reduce dental stain and formulation astringency, and be capable of reducing dental calculus. An effective amount of MSA will typically be from about 0.05% to about 35%, preferably from about 1% to about 30%, more preferably from about 5% to about 25%, and most preferably from about 6% to about 20%, by weight of the total oral composition.

In addition to binding stannous ions effectively, some of the MSA/chelating agents have been found useful as solubilizing agents for insoluble components of the composition. For example, Glass H polyphosphate has been found to solubilize insoluble stannous salts as well as stannous oxides and hydroxides.

Polyvalent Cation Source

The present compositions preferably comprise a source of polyvalent cations other than stannous, including inorganic ions such as zinc (Zn⁺²), copper (Cu⁺²), aluminum (Al⁺³), iron (Fe⁺², Fe⁺³), strontium (Sr⁺²), calcium (Ca⁺²), barium (Ba⁺²), magnesium (Mg⁺²) and manganese (Mn⁺²). Such polyvalent cations compete with stannous for the MSA/chelant incorporated in the compositions to stabilize stannous by binding or chelation. It has been found that while chelation is effective to stabilize stannous, such chemically stabilized stannous ion can have very limited bioavailability and thus, reduced therapeutic efficacy. By having competing cations in the same system with stannous, over-stabilization of stannous with the chelant is avoided, resulting in availability of stannous upon exchange with the competing cation(s). The inclusion of the second polyvalent cation thus enables the use of chelants such as low molecular weight diphosphonates, pyrophosphate and tripolyphosphate which have the ability to chelate stannous but tend to decrease therapeutic efficacy of stannous.

The polyvalent cation source is typically present at a level sufficient to supply a molar ratio of polyvalent cation to stannous preferably 1:1 or greater. This ensures a sufficient concentration of other polyvalent cation to compete with stannous for the MSA. The level of other polyvalent cations is of course also dependent on secondary considerations such as aesthetics and stability of the compositions and may be lower than the preferred 1:1 ratio. Preferred polyvalent cations are inorganic cations supplied from salts such as nitrate, chloride, fluoride, phosphate, pyrophosphate, polyphosphate, sulfate, carbonate, citrate, lactate, and oxalate or from oxides or hydroxides. The polyvalent cation source may be water soluble, sparingly-soluble or insoluble.

In a preferred embodiment, the composition comprises stannous fluoride as a source of stannous, a zinc salt as a source of zinc ions and a polyphosphate as MSA/chelant. The composition may be formulated as a single phase product or a two-phase product with stannous fluoride and the zinc salt in a first composition and the polyphosphate MSA in a second composition. In another embodiment of a dual phase composition, the second composition comprises a copolymer of maleic anhydride or acid and methyl vinyl ether (Gantrez) as MSA. In these embodiments, the preferred molar ratio of zinc ions to stannous ions ranges from about 0.5:1 to about 5:1.

In a further embodiment, the composition, which may be single-phase or dual-phase, comprises a calcium salt as a source of calcium ions, stannous fluoride as the source of stannous and fluoride and as MSA, a polyphosphate, a copolymer of maleic anhydride or acid and methyl vinyl ether or mixtures thereof. A dual-phase composition comprises stannous fluoride in a first composition and calcium chloride and MSA in a second composition.

Orally Acceptable Carrier Materials

In preparing the present compositions, it is desirable to add one or more carrier materials or excipients to the compositions. Such materials are well known in the art and are readily chosen by one skilled in the art based on the physical, aesthetic and performance properties desired for the compositions being prepared. These carriers may be included at levels typically from about 50% to about 99%, preferably from about 70% to about 98%, and more preferably from about 90% to about 95%, by weight of the oral composition. Examples of such optional carriers are described in the following paragraphs.

Total Water Content

Water employed in the preparation of commercially suitable oral compositions should preferably be of low ion content and free of organic impurities. In the oral composition, water may comprise from 0% up to about 95%, and preferably from about 5% to about 50%, by weight of the composition herein. This water content may be in a single phase oral composition or may be the resulting total water content of a dual phase oral composition. If the oral composition comprises a polyphosphate having an average chain length of about 4 or more, the composition or phase containing the polyphosphate will comprise a lower level of water, generally up to about 20% total water. Preferably, the total water content is from about 2% to about 20%, more preferably from about 4% to about 15%, and most preferably from about 5% to about 12%, by weight of the oral composition. The amounts of water include the free water which is added plus that which is introduced with other materials, such as with sorbitol, silica, surfactant solutions, and/or color solutions.

Buffering Agent

The present compositions may contain a buffering agent. Buffering agents, as used herein, refer to agents that can be used to adjust the pH of the compositions to a range of about pH 3.0 to about pH 10. The, phase of the oral composition containing stannous will typically have a slurry pH of from about 3.0 to about 7.0, preferably from about 3.25 to about 6.0, and more preferably from about 3.5 to about 5.5. The phase containing the MSA/chelating agent will typically have a slurry pH of from about 4.0 to about 10, preferably from about 4.5 to about 8, and more preferably from about 5.0 to about 7.0. An oral composition containing both stannous and a MSA/chelating agent in a single phase will typically have a pH of from about 4 to about 7, preferably from about 4.5 to about 6.5, and more preferably from about 5 to about 6.

Suitable buffering agents include alkali metal hydroxides, carbonates, sesquicarbonates, borates, silicates, phosphates, imidazole, and mixtures thereof. Specific buffering agents include monosodium phosphate, trisodium phosphate, sodium benzoate, benzoic acid, sodium hydroxide, potassium hydroxide, alkali metal carbonate salts, sodium carbonate, imidazole, pyrophosphate salts, citric acid, and sodium citrate. Preferred buffers would be those that control the pH in the target range without complexing stannous ions. Preferred buffering agents include acetic acid, sodium acetate, citric acid, sodium citrate, benzoic acid and sodium benzoate. Buffering agents are used at a level of from about 0.1% to about 30%, preferably from about 1% to about 10%, and more preferably from about 1.5% to about 3%, by weight of the present composition.

Fluoride Ion Sources

The oral compositions of the present invention will optionally include a soluble fluoride source capable of providing bioavailable and efficacious fluoride ions. Soluble fluoride ion sources include sodium fluoride, stannous fluoride, indium fluoride, amine fluoride and sodium monofluorophosphate. Stannous fluoride is a preferred soluble fluoride source. This ingredient may serve as both a/the stannous source and fluoride source. Norris et al., U.S. Pat. No. 2,946,725, issued Jul. 26, 1960, and Widder et al., U.S. Pat. No. 3,678,154 issued Jul. 18, 1972, disclose such fluoride sources as well as others.

The present compositions may contain a soluble fluoride ion source capable of providing from about 50 ppm to about 3500 ppm, and preferably from about 500 ppm to about 3000 ppm of free fluoride ions. To deliver the desired amount of fluoride ions, fluoride ion sources may be present in the total oral composition at an amount of from about 0.1% to about 5%, preferably from about 0.2% to about 1%, and more preferably from about 0.3% to about 0.6%, by weight of the total composition delivered to the oral cavity.

Anticalculus Agent

Materials known to be effective in reducing mineral deposition related to calculus formation may also be used herein as the MSA/chelating agent or stabilizer for stannous. Chelating agents are able to complex calcium found in the cell walls of the bacteria and can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus can be used for stannous stabilization.

Such chelating agents useful for their anticalculus activity include pyrophosphates, tripolyphosphates, and diphosphonates such as EHDP and AHP. The pyrophosphate salts useful as a source of pyrophosphate ion in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, Third Edition, Volume 17, Wiley-Interscience Publishers (1982).

Other examples of chelating agents used as anticalculus agent include ethylenediaminetetraacetic acid, nitrilotriacetic acid and related compounds disclosed in British Patent 490,384, Feb. 15, 1937; polyphosphonates in U.S. Pat. No. 3,678,154, Jul. 18, 1972 to Widder et al., U.S. Pat. No. 5,338,537 issued to Aug. 16, 1994 to White, Jr., and U.S. Pat. No. 5,451,401 issued Sep. 19, 1995 to Zerby et al.; carbonyl diphosphonates in U.S. Pat. No. 3,737,533, Jun. 5, 1973 to Francis; a zinc-polymer combination formed by the reaction or interaction of a zinc compound with an anionic polymer containing carboxylic, sulfonic and/or phosphonic acid radicals in U.S. Pat. No. 4,138,477, issued Feb. 6, 1979, to Gaffar; tartaric acid in U.S. Pat. No. 5,849,271 issued Dec. 15, 1998 and U.S. Pat. No. 5,622,689 issued Apr. 22, 1997 both to Lukacovic; acid or salt form of tartrate monosuccinate, tartrate disuccinate, and mixtures thereof in U.S. Pat. No. 5,015,467 issued May 14, 1991 to Smitherman; acrylic acid polymer or copolymer in U.S. Pat. No. 4,847,070, Jul. 11, 1989 to Pyrz et al. and in U.S. Pat. No. 4,661,341, Apr. 28, 1987 to Benedict et al.; sodium alginate in U.S. Pat. No. 4,775,525, issued Oct. 4, 1988, to Pera; polyvinyl pyrrolidone in GB 741,315 published Nov. 30, 1955, WO 99/12517 published Mar. 18, 1999 and U.S. Pat. No. 5,538,714 issued Jul. 23, 1996 to Pink et al.; and copolymers of vinyl pyrrolidone with carboxylates in U.S. Pat. No. 5,670,138 issued Sep. 23, 1997 to Venema et al. and in JP Publication No. 2000-0633250 to Lion Corporation, published Feb. 29, 2000. Other chelating agents that may be used as anticalculus agents include gluconic acid, tartaric acid, citric acid and pharmaceutically-acceptable salts thereof. Examples include sodium or potassium gluconate and citrate; citric acid/alkali metal citrate combination; zinc citrate trihydrate; disodium tartrate; dipotassium tartrate; sodium potassium tartrate; sodium hydrogen tartrate; potassium hydrogen tartrate. The amounts of such chelating agents suitable for use in the present invention are about 0.1% to about 2.5%, preferably from about 0.5% to about 2.5% and more preferably from about 1.0% to about 2.5%.

Still other anticalculus agents suitable for use in the present invention are the polymeric polycarboxylates disclosed in U.S. Pat. No. 4,138,477, Feb. 6, 1979 and U.S. Pat. No. 4,183,914, Jan. 15, 1980 to Gaffar et al. and include copolymers of maleic anhydride or acid with another polymerizable ethylenically unsaturated monomer, such as methyl vinyl ether (methoxyethylene), styrene, isobutylene or ethyl vinyl ether. Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride with methyl vinyl ether having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Abrasive Polishing Materials

An abrasive polishing material may also be included in the oral compositions. The abrasive polishing material contemplated for use in the compositions of the present invention can be any material which does not excessively abrade dentin. Additionally, the abrasive polishing material should be formulated in the oral composition so that it does not compromise the stability of the stannous or fluoride. For example, in a dual phase oral composition, the abrasive polishing material is preferably in a separate phase from the fluoride ion source and stannous ion source.

Typical abrasive polishing materials include silicas including gels and precipitates; aluminas; phosphates including orthophosphates, polymetaphosphates, and pyrophosphates; and mixtures thereof. Specific examples include dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510, issued Dec. 25, 1962. Mixtures of abrasives may also be used. If the oral composition or particular phase comprises a polyphosphate having an average chain length of about 4 or more, calcium containing abrasives and alumina are not preferred abrasives. The most preferred abrasive is silica.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, “Zeodent”, particularly the silica carrying the designation “Zeodent 119”. The types of silica dental abrasives useful in the toothpastes of the present invention are described in more detail in Wason, U.S. Pat. No. 4,340,583, issued Jul. 29, 1982. Other suitable silica abrasives are described in Rice, U.S. Pat. Nos. 5,589,160; 5,603,920; 5,651,958; 5,658,553; 5,716,601 and in White, Jr., et al. U.S. Pat. No. 6,740,311. The abrasive in the oral composition compositions described herein is generally present at a level of from about 6% to about 70% by weight of the composition. Preferably, oral compositions contain from about 10% to about 50% of abrasive, by weight of the oral composition.

Peroxide Source

The present invention may include a peroxide source in the composition. The peroxide source is selected from the group consisting of hydrogen peroxide, calcium peroxide, urea peroxide, and mixtures thereof. The preferred peroxide source is calcium peroxide. Preferably, to maximize stability, the peroxide source is not in the same phase as the stannous ion source. The following amounts represent the amount of peroxide raw material, although the peroxide source may contain ingredients other than the peroxide raw material. The present composition may contain from about 0.01% to about 10%, preferably from about 0.1% to about 5%, more preferably from about 0.2% to about 3%, and most preferably from about 0.3% to about 0.8% of a peroxide source, by weight of the oral composition.

Alkali Metal Bicarbonate Salt

The present invention may also include an alkali metal bicarbonate salt. Alkali metal bicarbonate salts are soluble in water and unless stabilized, tend to release carbon dioxide in an aqueous system. Sodium bicarbonate, also known as baking soda, is the preferred alkali metal bicarbonate salt. The alkali metal bicarbonate salt also functions as a buffering agent. Because of the pH at which alkali metal bicarbonate salts buffer, the bicarbonate salt is preferably in a phase separate from the stannous ion source. The present composition may contain from about 0.5% to about 50%, preferably from about 0.5% to about 30%, more preferably from about 2% to about 20%, and most preferably from about 5% to about 18% of an alkali metal bicarbonate salt, by weight of the oral composition.

Additional Carriers

The present invention compositions are in the form of toothpastes, dentifrices, topical oral gels, mouthrinse, denture product, mouthsprays, lozenges, oral tablets or chewing gums and typically contain some thickening material or binders to provide a desirable consistency. The amount and type of the thickening material will depend upon the form of the product. Preferred thickening agents are carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents can be used in an amount from about 0.1% to about 15%, by weight of the oral composition.

Another optional component of the compositions desired herein is a humectant. The humectant serves to keep oral compositions from hardening upon exposure to air and certain humectants can also impart desirable sweetness of flavor to toothpaste compositions. Suitable humectants for use in the invention include glycerin, sorbitol, polyethylene glycol, propylene glycol, xylitol, and other edible polyhydric alcohols. The humectant generally comprises from about 0% to 70%, and preferably from about 15% to 55%, by weight of the oral composition.

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof. Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. Nonionic surfactants which can be used in the compositions of the present invention can be broadly defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name Pluronic), polyoxyethylene, polyoxyethylene sorbitan esters (sold under trade name Tweens), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials. The nonionic surfactant poloxamer 407 is one of the most preferred surfactant because the poloxamer has been discovered to help reduce the astringency of the stannous. The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed. Many of the suitable nonionic and amphoteric surfactants are disclosed by Gieske et al., U.S. Pat. No. 4,051,234, issued Sep. 27, 1977. The present composition typically comprises one or more surfactants each at a level of from about 0.25% to about 12%, preferably from about 0.5% to about 8%, and most preferably from about 1% to about 6%, by weight of the composition.

Titanium dioxide may also be added to the present composition. Titanium dioxide is a white powder which adds opacity to the compositions. Titanium dioxide generally comprises from about 0.25% to about 5%, by weight of the composition.

Coloring agents may also be added to the present composition. The coloring agent may be in the form of an aqueous solution, preferably 1% coloring agent in a solution of water. Color solutions generally comprise from about 0.01% to about 5%, by weight of the composition.

A flavor system can also be added to the compositions. Suitable flavoring components include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) and mixtures thereof. A flavor system is generally used in the compositions at levels of from about 0.001% to about 5%, by weight of the composition.

Sweetening agents can be added to the compositions. These include saccharin, dextrose, sucrose, lactose, xylitol, maltose, levulose, aspartame, sodium cyclamate, D-tryptophan, dihydrochalcones, acesulfame, and mixtures thereof. Various coloring agents may also be incorporated in the present invention. Sweetening agents and coloring agents are generally used in toothpastes at levels of from about 0.005% to about 5%, by weight of the composition.

The present invention may also include other agents in addition to the stannous to provide antimicrobial benefits. These agents may be included at levels which do not prevent the interaction between stannous and the MSA. Included among such antimicrobial agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, among others. Triclosan monophosphate is an additional water soluble antimicrobial agent. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are examplary of typical quaternary ammonium antibacterial agents. Other compounds are bis[4-(R-amino)-l-pyridinium] alkanes as disclosed in U.S. Pat. No. 4,206,215, issued Jun. 3, 1980, to Bailey. Other antimicrobials such as copper bisglycinate, copper glycinate, zinc citrate, and zinc lactate may also be included. Also useful are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234, to Gieske et al. Specific antimicrobial agents include chlorhexidine, triclosan, triclosan monophosphate, and flavor oils such as thymol. Triclosan and other agents of this type are disclosed in U.S. Pat. No. 5,015,466, issued to Parran, Jr. et al. and U.S. Pat. No. 4,894,220, to Nabi et al. The water insoluble antimicrobial agents, water soluble agents, and enzymes may be present in either the first or second oral compositions if there are two phases. These agents may be present at levels of from about 0.01% to about 1.5%, by weight of the oral composition.

A dentifrice composition may be a paste, gel, or any configuration or combination thereof. If a dual phase dentifrice is desired, the first and second dentifrice compositions will be physically separated in a dentifrice dispenser. It is generally preferred that the first dentifrice composition be a paste and the second dentifrice composition be a gel. The dispenser may be a tube, pump, or any other container suitable for dispensing toothpaste. Dual compartment packages suitable for this purpose are described in U.S. Pat. No. 4,528,180; U.S. Pat. No. 4,687,663; and U.S. Pat. No. 4,849,213, all to Shaeffer. The dispenser will deliver approximately equal amounts of each dentifrice composition through an opening. The compositions may intermix once dispensed. Alternatively, the oral formulation may be delivered from a kit containing two separate dispensers which are used to deliver two dentifrice compositions that are both used simultaneously.

### Efficacy Measures

Overall performance of the present compositions may be defined in terms of an efficacy score/stain score ratio, wherein efficacy is measured using the in vitro Plaque Glycolysis and Regrowth Model (i-PGRM), and stain is measured using the in vitro Pellicle Tea Stain Model (i-PTSM). The present compositions provide an efficacy score to stain score ratio of at least 1.2, which represents a realistic improvement in that sufficient therapeutic efficacy is maintained while achieving a reduction in staining. Improvement in formulation astringency is defined as greater than 50% increase in formulation mouth feel parameters such as dry mouth, and clean mouth indices as defined in controlled consumer testing. Effectiveness for control of supragingival calculus is defined by activity in prevention of plaque calcification using the Modified Plaque Growth and Mineralization assay.

Antimicrobial Activity

The stannous ion concentration and bioavailability required for the provision of therapeutic actions may differ for different clinical actions, for example, caries vs. gingivitis. However, it is critical to establish a minimum antimicrobial activity level, since the therapeutic activity of stannous can be compromised below this level. It is especially important to maintain efficacy in compositions wherein binding of stannous occurs, since stannous binding can easily lead to loss of antimicrobial activity. Herein, the minimum efficacy provided by the stannous ion source is defined in terms of effects in producing metabolic inhibition of dental plaque bacterial biofilms, which are responsible for numerous undesirable intraoral conditions. Efficacy is thus defined in terms of a noticeable and significant reduction in in situ plaque metabolism as measured using the in vitro Plaque Glycolysis and Regrowth Model (i-PGRM), developed in our laboratories. The i-PGRM has been demonstrated to provide an excellent correlation to bioavailability of stannous fluoride required to produce clinical antimicrobial, antigingivitis and antiplaque activity of oral compositions containing stannous fluoride. The efficacy of stannous containing compositions for gingivitis can be directly compared to a stannous-containing dentifrice formulation such as described in U.S. Pat. No. 5,004,597 to Majeti, et al. or to a marketed dentifrice containing stannous fluoride, Crest Gum Care.

The i-PGRM is a technique where plaque is grown from human saliva, and treated with agents designed to produce various levels of antimicrobial activity. The purpose of this technique is to provide a simple and quick method for determining if compounds have a direct effect on the metabolic pathways that plaque microorganisms utilize for the production of toxins which adversely affect gingival health. In particular, the model focuses on the production of organic acids including lactic, acetic, propionic, and butyric. This method utilizes plaque grown on polished glass rods which have been dipped in saliva overnight, soy broth and sucrose for 6 hours, and saliva again overnight. The plaque mass grown on the glass rods is then treated for 1 minute with a 3:1 water to dentifrice slurry. The mass is then placed in a soy broth/sucrose solution for 6 hours and the pH of the incubation solution is measured at the end of the 6 hours. Thus, there are measures of pre-incubation pH and post incubation pH for both test formulations and controls. This testing is typically done with a number of replicates to minimize experimental variances, and a mean pH is calculated from the replicates. Due to strong reactivity with saccharolytic organisms, compositions containing high levels of bioavailable stannous produce significant inhibition of plaque acid generation in the i-PGRM assay. This enables formulation variations to be compared for stability and bioavailability of stannous with relative ease.

Stannous fluoride and/or other stannous salts are found in the oral compositions described herein in an effective amount to provide a desired i-PGRM score. The desired i-PGRM score is measured relative to non-stannous containing formulations (negative control) and to stannous-containing formulations (positive control) such as described in U.S. Pat. No. 5,004,597 to Majeti et al. Most preferable i-PGRM scores are significantly different from placebo controls and ideally similar to those provided by conventional stannous fluoride compositions proven effective for reducing plaque and gingivitis. Research has demonstrated that effective gingivitis efficacy can be anticipated for compositions providing at least about 60%, preferably at least about 70%, and more preferably at least about 80% of an effective stannous-containing dentifrice such as shown in Example II, Comparative Example below.

The i-PGRM score is calculated according to the formula:

$$i \text{-} PGRM Score = 100 \% \times \frac{\begin{matrix}( Test product mean pH - \\ Non \text{-} Stannous Control mean pH )\end{matrix}}{\begin{matrix}( Stannous Control mean pH - \\ Non \text{-} Stannous Control mean pH )\end{matrix}}$$

The mean pH values refer to incubation media pH's obtained following treatment and sucrose challenge. The non-stannous control plaque samples produce large amounts of acid, and hence their pH's are lower than that of plaque samples treated with the positive control (stabilized stannous fluoride dentifrice as shown in Example II, Comparative Example). The effectiveness of a formulation prepared from the combination of a stannous ion source and MSA will ideally be comparable to the stannous-containing control, and hence ideal i-PGRM score should approach 100%.

Staining Reduction

Tooth staining is a common undesirable side effect of the use of stannous fluoride compositions. Improved stannous fluoride dentifrices described herein provide reduced dental stain formation resulting from more efficient stannous delivery from stannous bound to the MSA. The staining of the tooth surface typically caused by stannous is measured in the clinical situation by using a stain index such as the Lobene or Meckel indices described in the literature. An in vitro staining model has also been developed which provides quantitative estimates for stannous fluoride formulation staining potential which correlate well with clinical observations. Formulations can thus be tested in advance of clinical examination using these methods.

The in-vitro Pellicle Tea Stain Model (i-PTSM) is a technique where an in vitro plaque biomass is grown on glass rods from pooled human stimulated saliva over the course of three days. The plaque biomass is treated with 3:1 water to dentifrice supernatants, where abrasive and insoluble solids have been removed via centrifugation, to determine potential dental staining levels of the various agents. The purpose of this technique is to provide a simple and quick method for determining if compounds have a direct effect on the amount of dental plaque stain. This method utilizes plaque grown on polished glass rods from pooled human saliva with treatments of 5 minutes each, followed by a 10 minute tea treatment. The treatment regimen is repeated at lest three times before the plaque mass is digested off the rods, filtered and absorbance at 380 nm is measured. This testing is typically done with a number of replicates to minimize experimental variances, and a mean absorbance is calculated from the replicates.

It has been found that the stain, which is typically produced by effective stannous fluoride is reduced by combining the stannous fluoride with one or a mixture of the MSA's discussed above. The benefit of reducing the staining caused by stannous is achieved with the present compositions without significantly compromising the efficacy of the stannous, fluoride, and MSA. The amount of staining resulting from the oral compositions of the present invention is significantly lower than the amount of staining resulting from typical dentifrices containing stannous. The term “reduced” as used herein means a statistically significant reduction. Therefore, reducing the staining of stannous means that the amount of stain is statistically significantly reduced compared to a stannous-containing positive control. Not reducing the efficacy of the stannous means the efficacy of the stannous is not statistically significantly reduced relative to a stannous-containing positive control. Alternatively, stain may be measured relative to typical oral compositions, which do not contain stannous fluoride or another antimicrobial agent which is known to stain. Therefore, the compositions may be measured relative to very little to no stain.

The i-PTSM score can be calculated from this staining assay according to the formula:

$$i \text{-} PTSM Score = 100 \% \times \frac{Test Product Mean Absorbance}{( Stannous Control Mean Absorbance )}$$

The mean absorbance values refer to digested plaque calorimetric values obtained following dentifrice treatments and tea rinsing challenge. The stannous control used is typically a high staining stannous fluoride formulation such as illustrated in Example II, Comparative Example below. The stannous control samples produce large amounts of tea absorption and hence increased colorimetric absorbance. Thus, the i-PTSM score is a measure of the relative level of staining. The lower the score, the lower the level of staining. The combination of a stannous ion source and MSA provides a reduction in staining and will ideally have a i-PTSM score of less than about 75%, preferably less than 60%, more preferably less than 50%, most preferably less than 25%.

Ratio of i-PGRM Score to i-PTSM Score

A key descriptor of the improvement in stannous compositions provided herein is the ratio of efficacy of stannous in comparison to staining potential, these being key consumer concerns. The effectiveness of the oral composition of the present invention will be measured by a ratio of i-PGRM score to i-PTSM score.

The ratio of i-PGRM score to i-PTSM score is calculated according to the formula:
Ratio=i-PGRM score/i-PTSM score

In accordance with the present invention, the ratio developed using these methods should be at least about 1.2 for significant improvements in stannous formulation efficacy relative to tooth staining side effects. The ratio is preferably above about 1.3, more preferably above about 1.5, and most preferably above about 2.0. If there is little to no stain occurring, the ratio approaches infinity, which is preferred.

Binding of Stannous

As discussed above, effective stabilization of stannous (efficacy with reduced side effects) may be accomplished by in situ binding or complexation of stannous ion with the mineral surface active agent (MSA). In mixed compositions containing stannous fluoride, evidence of binding of stannous is readily observed by potentiometric detection of available ionic fluoride. For example, binding of stannous with polyphosphate MSA ligand results in exchange of fluoride from stannous fluoride and release as ionic fluoride into solution. Relevant measures of stannous binding can be assessed by this technique because fluoride is the strongest ligand in the system after the MSA binding agent. Thus, fluoride release is illustrative of stannous binding by the MSA under these conditions.

This approach was employed to determine the complexation of stannous and zinc ions with polyphosphate MSA. A fluoride release assay using the ion selective fluoride electrode was used to monitor the release and complexation of fluoride by stannous ion as it is being complexed by polyphosphate anion. In a solution of stannous fluoride, the addition of Glass H polyphosphate produces free fluoride ion because of stannous-polyphosphate complex formation and subsequent release of fluoride ion from stannous fluoride complexes. Since zinc ion may have a higher affinity than stannous ion for polyphosphate anion, the addition of zinc salt into a mixture of stannous fluoride and polyphosphate would favor the formation of zinc-polyphosphate complex and release stannous ions that will subsequently complex free fluoride. Thus the level of free fluoride ion will be lowered by the addition of zinc ion.

Solutions containing stannous fluoride alone and stannous fluoride plus zinc salt were prepared. To these solutions glass H polyphosphate solution were added in increments and fluoride ion monitored via fluoride ion selective electrode. The following two solutions were prepared.

Solution 1: SnF₂ 0.454%

Solution 2: SnF₂ 0.454% +ZnCl₂ 1.25% (6000 ppm of zinc ion)

To each of these solutions 0.1M Glass H polyphosphate (22% w/v) was titrated and fluoride ion measured. The data are plotted as free fluoride concentration as a function of the amount of titrant added. The results of this in vitro metal-ligand complexation study are presented in the table below. Theoretical fluoride release from 0.454% stannous fluoride would be 1100 ppm free fluoride. Since most of the fluoride in a solution containing stannous fluoride is complexed with stannous ion, there will be very little free fluoride. With the addition of polyphosphate the free fluoride ion increases significantly and approaches the theoretical value of 1100 ppm. This is due to complexation of stannous fluoride with polyphosphate anion to form stannous-polyphosphate complex with simultaneous release of fluoride ion from stannous fluoride complexes. The addition of zinc ion in the stannous fluoride solution reduces the level of free fluoride ion with increase in polyphosphate level. This indicates that zinc ion helps liberate stannous ion from stannous—polyphosphate complex by competitively binding with polyphosphate anion.

| | Free Ionic |
|---|---|
| Solution Composition | Fluoride (ppm) |
| SnF₂ (0.454%) | 29 |
| SnF₂ (0.454%) Mixed with Polyphosphate | 669 |
| (6.2%) | |
| SnF₂ (0.454%) + ZnCl₂ (1.2%) + Polyphosphate | 355 |
| (6.2%) | |

Astringency Reduction

Astringency is an additional side effect of many stannous containing compositions which is significantly improved in the present compositions comprising the MSA's in combination with stannous and a second polyvalent cation. The astringency of formulations can be measured in intraoral panels, where subjects assess mouth condition before and after tooth brushing with the test formulations. In these studies, time dependent studies can be made of dentifrice effects on consumer subjective responses. In one protocol, panelists began a conditioning series by having teeth cleaned with vigorous self oral hygiene including brushing for two three minute periods, flossing and disclosing to ensure complete plaque removal. Subjects are then assigned their test product and instructed to brush with twice per day as usual. For these tests, subjects reported in the morning to a clinic prior to any oral hygiene or food or beverage consumption. Panelists are then asked to fill out a subjective mouth feel assessment questionnaire including questions on tooth clean feeling, smooth teeth feeling and clean mouth feeling as well as assessments of mouth moisture. Panelists then brushed for one minute with assigned oral product. At this point, before lunch and before dinner (late p.m.) subjects again filled out subjective mouth feel questionnaire. Results of these tests show that the present formulations containing stannous salts in combination with a MSA such as Glass H polyphosphate produce a marked improvement in formulation astringency post brushing. Astringency is reduced compared to conventional stannous formulations without the MSA. Acceptability of the present formulation is comparable to conventional sodium fluoride (NaF) and tartar control dentifrices respectively.

Reduction and Control of Calculus

The provision of anticalculus benefits is another desirable aspect of the present stannous fluoride formulations. Anticalculus activity can be predicted from mineral surface activity measurements and the application of plaque growth and mineralization assays. The present compositions include MSA's, such as polyphosphates that bind stannous ions. Preferred compositions contain MSA phosphate polymers with significant affinity for dental surfaces, which are comprised of calcium hydroxyapatites. Preferred MSA's will include phosphate polymers which produce significant reductions in calcium phosphate mineralization as established in controlled mineralization assays. Polyphosphates (in particular linear polyphosphates with average chain lengths greater than about 4) have been found to produce superior activity and substantivity to oral surfaces compared to pyrophosphate and some other commonly used dental cleaning ingredients. The increased activity and substantivity translate into significant improvements in the prevention of dental stains and supragingival calculus and in the non-abrasive removal of dental stains. Without wishing to be bound by theory, it is believed that the polyphosphates prevent formation of supragingival calculus by essentially disrupting the mineralization process, which is the formation of hard calcium phosphate mineral deposits on tooth enamel. By binding to tooth surfaces, polyphosphates disrupt the mineral building process, because their structures do not adequately fit the developing mineral lattice, which becomes the calculus.

Method of Treatment

The present invention also relates to a method of treating gingivitis and plaque with reduced staining, by using the present compositions comprising a stannous ion source, a source of polyvalent metal ions other than stannous and mineral surface-active/chelating agent such as a linear polyphosphate having an average chain length of about 4 or more. A source of fluoride ions is preferably included in the compositions particularly if the stannous ion source does not comprise stannous fluoride or stannous fluorophosphate. Additionally provided are methods of providing oral care compositions, which have caries, gingivitis, plaque, tartar, stain, sensitivity, aesthetics, breath, mouthfeel, and cleaning benefits. The benefits of these compositions may increase over time when the composition is repeatedly used. Specifically, the method of treatment will include reducing the gingivitis and plaque, as measured by the i-PGRM, while reducing the staining caused by oral composition containing stannous, as measured by the i-PTSM. The ratio of the i-PGRM score to i-PTSM stain model score is above about 1.2.

The present invention also relates to methods for reducing the incidence of calculus on dental enamel and to methods for providing desirable mouth aesthetic benefits including reduced astringency and oral surface conditioning effects. The benefits of these compositions may increase over time when the composition is repeatedly used.

Methods of treatment include preparing an oral composition containing a stannous ion source, a source of polyvalent cation other than stannous and a mineral surface active agent (MSA) and administering the composition to the subject. Administering to the subject may be defined as having the oral care composition contact the tooth surfaces of the subject by brushing with a dentifrice or rinsing with a dentifrice slurry. Administration may also be by contacting the topical oral gel, mouthrinse, denture product, mouthspray, oral tablet, lozenge, or chewing gum with the tooth surfaces. The subject may be any person or animal in need of treatment or prevention of oral conditions including plaque, gingivitis, tartar, stain, and sensitivity. By “animal” is meant to include in particular household pets or other domestic animals, or animals kept in captivity.

For example, a method of treatment may include a person brushing a dog's teeth with one of the dentifrice compositions. Another example would include the rinsing of a cat's mouth with an oral composition for a sufficient amount of time to see a benefit. Pet care products such as chews and toys may be formulated to contain the present oral compositions. The composition including stannous, second cation and MSA is incorporated into a relatively supple but strong and durable material such as rawhide, ropes made from natural or synthetic fibers, and polymeric articles made from nylon, polyester or thermoplastic polyurethane. As the animal chews, licks or gnaws the product, the incorporated active elements are released into the animal's oral cavity into a salivary medium, comparable to an effective brushing or rinsing.

### EXAMPLES

The following examples and descriptions further clarify embodiments within the scope of the present invention. These examples are given solely for the purpose of illustration and are not to be construed as limitations of the present invention as many variations thereof are possible without departing from the spirit and scope.

### Example I

Example I illustrates dual phase dentifrice compositions incorporating stannous fluoride and/or other stannous salts in a First Dentifrice composition and incorporating a MSA such as sodium polyphosphate (Glass H supplied by FMC Corporation, n=21 condensed phosphate polymer) or copolymers of maleic anhydride or acid and methyl vinyl ether (Gantrez) in a Second Dentifrice composition. The second polyvalent cation source is incorporated in either composition. These compositions may be suitably prepared by conventional methods chosen by the formulator.

First Dentifrice Compositions

| Ingredient | 1a | 2a | 3a | 4a | 5a |
|---|---|---|---|---|---|
| Stannous Fluoride | 1.062 | 1.062 | 1.062 | 1.062 | — |
| Stannous Chloride | 1.500 | — | — | — | 1.500 |
| Zinc Lactate | — | — | | 5.000 | — |
| Zinc Carbonate | — | — | 5.000 | — | — |
| Sodium Fluoride | — | — | — | — | 0.486 |
| Sodium Lauryl Sulfate | — | — | 2.500 | 2.500 | 7.500 |
| 27.9% soln. | | | | | |
| Sodium Gluconate | 3.290 | 1.300 | 2.940 | 1.840 | 4.135 |
| Sodium Hydroxide | 0.600 | 0.600 | 0.600 | 0.280 | 0.900 |
| 50% soln. | | | | | |
| Sodium Saccharin | 0.400 | 0.400 | 0.400 | 0.300 | 0.400 |
| Flavor | 1.500 | 1.300 | 1.300 | 1.200 | 1.100 |
| FD&C Blue #1 | 0.300 | 0.300 | 0.300 | 0.100 | 0.500 |
| Dye 1% soln. | | | | | |
| Polyethylene Glycol | — | — | — | — | 6.000 |
| Poloxamer 407 | 15.500 | 15.500 | 17.500 | 16.500 | 7.000 |
| Sorbitol, 70% soln. | 23.000 | 23.000 | — | — | 36.879 |
| Glycerin | 31.008 | 31.008 | 22.000 | 22.000 | 20.000 |
| Xanthan Gum | — | — | 0.850 | — | 1.100 |
| Hydroxyethyl Cellulose | — | — | — | — | 0.500 |
| Polyoxyethylene | — | — | 0.200 | — | — |
| Water | 21.840 | 25.530 | 45.348 | 49.218 | 12.000 |

Second Dentifrice Compositions

| Ingredient | 1b | 2b | 3b | 4b | 5b |
|---|---|---|---|---|---|
| Glass H Polyphosphate | 25.800 | 20.800 | 26.000 | — | 18.000 |
| Gantrez | — | 5.000 | — | 5.000 | — |
| Zinc Lactate | — | — | — | — | 5.000 |
| Calcium Chloride | 1.712 | 1.712 | — | — | — |
| Carboxymethycellulose | 0.500 | 0.500 | 0.200 | 0.200 | 0.300 |
| Water | 2.210 | 2.210 | — | 32.150 | 1.400 |
| Flavor | 1.500 | 1.500 | 1.100 | 1.100 | 1.100 |
| Glycerin | 28.178 | 28.178 | 45.550 | 20.550 | 34.850 |
| Polyethylene Glycol | 1.500 | 1.500 | — | — | 6.000 |
| Propylene Glycol | 8.000 | 8.000 | — | — | — |
| Polyoxyethylene | 0.200 | 0.200 | — | — | — |
| Sodium Lauryl Sulfate | 10.000 | 10.000 | 8.000 | 10.000 | 6.000 |
| 27.9% soln. | | | | | |
| Silica | 15.000 | 15.000 | 18.150 | 30.000 | 26.000 |
| Polyoxyl 40 | 2.500 | 2.500 | — | — | — |
| Hydrogenated Castor Oil | | | | | |
| Benzoic Acid | 0.600 | 0.600 | — | — | 0.300 |
| Sodium Benzoate | 0.600 | 0.600 | — | — | 0.300 |
| Sodium Saccharin | 0.400 | 0.400 | 0.400 | 0.400 | 0.350 |
| Titanium Dioxide | 1.000 | 1.000 | 0.500 | 0.500 | 0.400 |
| Xanthan Gum | 0.300 | 0.300 | 0.100 | 0.100 | — |

### EXAMPLE II

Example II illustrates single phase dentifrice compositions incorporating stannous salt(s) as stannous ion source, a polyvalent cation source, and Glass H sodium polyphosphate or Gantrez as MSA. The compositions may be prepared using conventional methods.

| Ingredient | A | B | C | D | E | F |
|---|---|---|---|---|---|---|
| Stannous | 0.454 | 0.454 | 0.454 | 0.454 | 0.454 | — |
| Fluoride | | | | | | |
| Stannous | | | | | | 1.500 |
| Chloride | | | | | | |
| Sodium | 13.000 | — | 13.000 | 7.000 | — | — |
| Polyphosphate | | | | | | |
| Gantrez | — | 2.000 | — | 2.000 | 2.000 | 2.000 |
| Zinc Lactate | 2.500 | 1.500 | — | 2.000 | — | 2.000 |
| Zinc carbonate | — | — | — | — | 2.000 | — |
| Calcium | — | — | 0.423 | — | — | |
| chloride | | | | | | |
| Sodium | — | — | — | — | — | 0.243 |
| fluoride | | | | | | |
| Silica | 20.000 | 20.000 | 20.000 | 20.000 | 20.000 | 20.000 |
| Propylene | 7.000 | 7.000 | 7.000 | 7.000 | — | — |
| Glycol | | | | | | |
| Polyethylene | 7.000 | 7.000 | 7.000 | 7.000 | — | — |
| Glycol | | | | | | |
| Poloxamer 407 | — | — | — | — | — | — |
| Sodium Lauryl | 3.400 | 3.400 | 3.400 | 3.400 | 4.300 | 4.300 |
| Sulfate | | | | | | |
| 27.9% soln. | | | | | | |
| Glycerin | 42.244 | 54.244 | 44.321 | 46.744 | 16.894 | 15.605 |
| Sorbitol 70% | — | — | — | — | 20.000 | 20.000 |
| soln. | | | | | | |
| Sodium | 1.100 | 1.100 | 1.100 | 1.100 | 1.100 | 1.100 |
| phosphate | | | | | | |
| tribasic | | | | | | |
| Flavor | 1.000 | 1.000 | 1.000 | 1.000 | 1.300 | 1.300 |
| Sodium | 0.652 | 0.652 | 0.652 | 0.652 | 0.652 | 0.652 |
| Gluconate | | | | | | |
| Carrageenan | 0.600 | 0.600 | 0.600 | 0.600 | — | — |
| Sodium | 0.500 | 0.500 | 0.500 | 0.500 | 0.500 | 0.500 |
| Saccharin | | | | | | |
| FD&C Blue #1 | 0.300 | 0.300 | 0.300 | 0.300 | 0.300 | 0.300 |
| 1% soln. | | | | | | |
| Xanthan Gum | 0.250 | 0.250 | 0.250 | 0.250 | 0.250 | 0.250 |
| Carboxymethyl | — | — | — | — | 0.250 | 0.250 |
| cellulose | | | | | | |
| Water | — | — | — | — | 30.000 | 30.000 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition having antimicrobial activity effective for reducing plaque and gingivitis, said composition comprising:
a. a stannous ion source,
b. a source of polyvalent cations other than stannous, and
c. a mineral surface-active agent having substantivity to teeth and chelating activity for stannous and said polyvalent cations,
said composition providing enhanced stannous stability and therapeutic efficacy.

2. An oral care composition according to claim 1 wherein the stannous ion source is selected from stannous fluoride, stannous chloride dihydrate, stannous acetate, stannous gluconate, stannous oxalate, stannous sulfate, stannous lactate, stannous tartrate, and mixtures thereof.

3. An oral care composition according to claim 1 wherein the polyvalent cation source provides polyvalent inorganic cations selected from zinc (Zn⁺²), copper (Cu⁺²), aluminum (Al⁺³), iron (Fe⁺², Fe⁺³), strontium (Sr⁺²), calcium (Ca⁺²), barium (Ba⁺²), magnesium (Mg⁺²), manganese (Mn⁺²), and mixtures thereof.

4. An oral care composition according to claim 1 wherein the mineral surface-active agent is polymeric and is a polyelectrolyte selected from phosphorylated polymers; polyphosphonates; polycarboxylates; carboxy-substituted polymers; copolymers of phosphate- or phosphonate-containing monomers or polymers with ethylenically unsaturated monomers, amino acids, proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); and mixtures thereof.

5. An oral care composition according to claim 4 comprising from about 0.5% to about 35% of a condensed phosphorylated polymer.

6. An oral care composition according to claim 5 wherein the phosphorylated polymer is a linear polyphosphate having an average chain length of from about 2 to about 125.

7. An oral care composition according to claim 4 wherein the mineral surface active agent comprises a copolymer of maleic anhydride or acid with methyl vinyl ether.

8. An oral care composition according to claim 4 wherein the mineral surface active agent comprises a diphosphonate/acrylate copolymer or cotelomer.

9. An oral care composition according to claim 1 further comprising materials selected from the group consisting of fluoride ion sources, antibacterial agents, surfactants, thickening materials, humectants, buffering agents, titanium dioxide, flavor systems, sweetening agents, coloring agents, and mixtures thereof.

10. An oral care composition according to claim 9 wherein the fluoride ion source is selected from the group consisting of sodium fluoride, stannous fluoride, indium fluoride, amine fluoride, sodium monofluorophosphate, and mixtures thereof.

11. An oral care composition according to claim 9 wherein the thickening material is selected from the group consisting of carboxyvinyl polymers, carrageenan, hydroxyethyl cellulose, sodium carboxymethylcellulose, sodium hydroxyethyl cellulose, gum karaya, xanthan gum, gum arabic, gum tragacanth, magnesium aluminum silicate, silica, and mixtures thereof.

12. An oral care composition comprising:
(a) a first composition comprising a stannous ion source,
(b) a second composition comprising a mineral surface-active agent having substantivity to mineral surfaces and chelating activity for stannous and said polyvalent cations, and
(c) a source of polyvalent cations other than stannous present in either or both of said first and second compositions,
wherein said mineral surface-active agent chelates the stannous ions upon intraoral contact of the first composition and second composition.

13. An oral care composition according to claim 12, wherein the stannous ion source comprises stannous fluoride, the polyvalent cation source comprises a zinc salt and the mineral surface-active agent comprises a polymer selected from linear polyphosphates having an average chain length of about 4 or more, copolymers of maleic anhydride or acid with methyl vinyl ether, and mixtures thereof.

14. A method of enhancing therapeutic efficacy of oral care compositions containing stannous for reducing plaque and gingivitis while decreasing staining and improving aesthetic desirability, comprising formulating said oral care compositions to comprise
a) a stannous ion source,
b) a source of polyvalent cations selected from zinc (Zn⁺²), copper (Cu⁺²), aluminum (Al⁺³), iron (Fe⁺², Fe⁺³), strontium (Sr⁺²), calcium (Ca⁺²), barium (Ba⁺²), magnesium (Mg⁺²), manganese (Mn⁺²), and mixtures thereof, and
c) a mineral surface-active agent having substantivity to teeth,
wherein said mineral surface-active agent binds stannous ions and said polyvalent cations resulting in increased stability and bioavailability of stannous.
